(19) United States
(12) Patent Application Publication
(10) Pub. No.: US 2003/0130184 A1
(43) Pub. Date: Jul. 10, 2003

(54) Methods of inducing cell death

(75) Inventors: Kevin Leon Gaston, Bristol (GB); Peter Leslie Stern, Stockport (GB); Anthony Russell Clarke, Bristol (GB)

Correspondence Address: Mark B. Wilson, FULBRIGHT & JAWORSKI L.L.P., Suite 2400, 600 Congress Avenue, Austin, TX, 78701, US

(73) Assignee: The University of Bristol, Bristol (GB)

(21) Appl. No.: 10/047,990

(22) Filed: Jan. 14, 2002

Related U.S. Application Data
(63) Continuation of application No. PCT/GB00/02693, filed on Jul. 13, 1999.

(30) Foreign Application Priority Data
Jul. 13, 1999 (GB) 9916363.6

(51) Int. Cl. A61K 38/17; C12P 21/02; C12N 5/06

(52) U.S. Cl. 514/12; 435/325

(57) ABSTRACT

This invention relates to methods of inducing cell death and preferably, though not exclusively to methods of killing cells using E2 and/or E7 proteins from papillomaviruses.

This invention relates to methods of inducing cell death and preferably, though not exclusively to methods of killing cells using E2 and/or E7 proteins from papillomaviruses.

Papillomaviruses (PV), viruses of the family Papoviridae, are DNA viruses which have double stranded circular DNA containing a number of genes including the E2, E6 and E7 genes. They infect epithelial cells and generally induce the formation of benign hyperproliferative lesions. Millions of men and women have a genital tract infection of one of at least 95 types of human papillomavinis (HPV) leading to genital warts. However, some papillomavirus types are associated with more serious conditions such as cancer. For example, HPV types 16 and 18 have been linked to cervical cancer in women (zur Hausen, H (1991) Virology, 184, 9-13) and bovine papillomavirus (BPV) types 2 and 4 have been linked to bladder cancer and cancer of the upper alimentary canal respectively, in cattle (Campo, M. S., et al (1992) Cancer Res. 52, 6898-6904, Campo, M. S., et al. (1994) Carcinogenesis, 15, 1597-1601). Human cervical cancer cells express the viral E6 and E7 oncogenes and the products of these genes increase cell proliferation and promote cell immortalisation (Crook, T., and Vousden, K. H. (1996) Papillomavirus Reviews: Current Research on Papillomaviruses (Lacey, C., ed) pp. 55-60, Leeds University Press, Leeds). The human papillomavirus E2 gene, or lack thereof is also thought to play a major role in the development of cervical cancer with PV-infected cells. Most cervical cancers contain chromosomally integrated copies of the HPV genome in which the viral E2 gene has been disrupted as a result of the opening of the viral circular DNA (Baker, C. C., et al. (1987) J. Virol., 61, 962-971). Furthermore, mutations in the E2 gene increase the immortalisation capacity of HPV16 (Romanczuk, H., and Howley, P. M. (1992) Proc. Natl. Acad. Sci. USA. 89. 3159-3163).

The papillomavirus E2 genes encode sequence-specific DNA binding proteins that regulate viral gene expression and which are also required for viral DNA replication (Thierry, F. (1996) Papillomavirns reviews: current research on papillomaviruses (Lacey, C., ed) pp. 21-29, Leeds University Press, Leeds). The E2 proteins bind as dimers to multiple copies of an inverted repeat sequence found within the viral long control region (LCR). Depending on the particular PV virus, and the particular E2 protein being studied, the binding of E2 to these sites can either activate or repress transciption of the E6 and E7 oncogenes. For example, the HPV16 E2 protein activates transcription from the P97 promoter located at the 3′ end of the HPV 16 LCR which causes increased transcription of the E6 and E7 oncogenes whereas, under exactly the same conditions, the BPV1 E2 protein represses P97 promoter activity (Boulvard, V., et al. (1994) EMBO J. 13, 5451-5459, Kovelman, R et al (1996) Virol, 70, 7549-7560). Each subunit of the E2 dimer contains two domains which are separated by a flexible hinge region: the N-terminal domain of each subunit mediates the regulation of viral transcription whereas the C-terminal domain mediates DNA binding (Giri, I., and Yaniv, M (1988) EMBO J., 7, 2823-2329). In BPV, truncated E2 proteins that lack the N-terminal transcriptional domain are also expressed. These truncated E2 proteins (E2-TR) can repress viral transciption and can also form transciptionally inactive heterodimers with full length E2 (Barsoum, J. et al. (1992) J. Virol., 66, 3941-3945).

The E2 proteins from HPV 16, HPV 18, and BPV1 all have dramatic effects on the proliferation and suival of cervical carcinoma cell lines. We have shown expression of the HPV 16 E2 protein in modified SiHa cells. SiHa cells are an HPV16-transformed cell line that contains a single disrupted copy of the E2 gene. We produced SiHa-E2 stable cell lines that express the HPV16 E2 protein under the control of the heavy metal-inducible metallothionein promoter. The induction of E2 expression in these cells using heavy metals results in cell death via apoptosis (Sanchez-Perez, A. et al. (1997) J. Gen. Virol., 78. 3009-3018). The E2-induced cell death showed several of the features characteristic of apoptosis including: blebbing of the plasma membrane, chromatin condensation, and the appearance of cell fragments with sub-G0 DNA content. Similarly, the HPV18 E2 protein induces apoptosis in HeLa cells, an HPV 18-tansformed cell line that also contains disrupted copies of the E2 gene (Desaintes, C., et al. (1997) EMBO J. 16. 504-514). Expression of the BPV1 E2 protein in either SiHa or HeLa cells has been shown to suppress proliferation, in part at least, by blocking the cells' transition from G1 to S phase (Hwang, E. S., et al (1993) J. Virol., 67, 3720-3729, Dowlanick, J. J., et al. (1995) J. Virol., 69. 7791-7799, Hwang, E. S., et al (1996) Oncogene, 12, 795-803). Since the proliferation assays used in these experiments scored colony formation after several days in culture, BPV1 E2 might also induce apoptosis in these cell lines.

There is also some evidence to suggest that the E2 proteins might have effects on non-HPV-transformed cells. Expression of the HPV 31 E2 protein in HPV-negative normal human foresidn keratinocytes (NHK cells) using a recombinant adenovirus, resulted in S-phase cell cycle arrest, and the appearance of cells with sub-G0 DNA content; a characteristic feature of apoptotic cell death (Frattini M. G., et al (1997) EMBO J., 16, 318-331). However, BPV1 E2 has no effect on the proliferation of C33a cells, an HPV-negative cervical carcinoma cell line, or SAOS cells, an HPV-negative osteosarcoma cell line (Dowhanick, J. J. et al. (1995) J. Virol. 69. 7791-7799). Furthermore, the HPV 18 E2 protein has no effect on the levels of apoptosis in C33a cells, SAOS cells, or HaCat cells, an HPV-negative spontaneously immortalised human keratinocyte cell line (Desaintes, C., et al (1997) EMBO J., 16. 504 514).

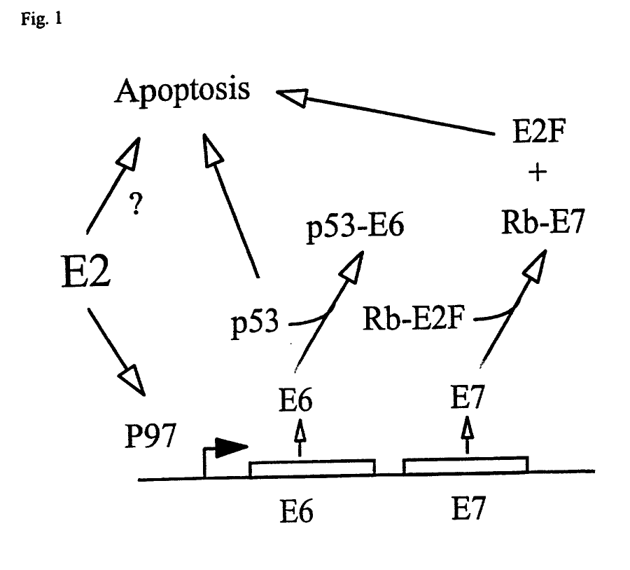

At present, there is no model that can explain satisfactorily the effects of the E2 proteins on cell proliferation. FIG. 1 is a schematic representation of some of the possible routes from the HPV 16 E2 protein to the induction of apoptosis. The bottom line represents the integrated HPV genome and the bent arrow indicates the P97 promoter. The E2 protein regulates siption of the HPV 16 E6 and E7 genes (open boxes). E6 binds to p53 and this reduces the half-life of p53 within the cell. E7 binds to Rb and brings about the release of E2F. Both p53 and E2F can bring about apoptosis. Note that E2 could also induce apoptosis independently of its effects on transciption of E6 and E7. In cell lines that contain integrated HPV DNA BPV1 E2 and HPV18 E2 have been shown to repress transcription of the HPV18 E6 and E7 oncogenes (Hwang, E. S. et al (1993) J. Virol 67. 3720-3729, Desaintes, C., et al (1997) EMBO J., 16. 504-514).

The tumour suppression protein p53 is well characterized. There are many mutants of p53 as well as p53-related genes such as p73 and p63 (White, E. and Prives. C., Nature, 399. June 1999). The E6 protein binds to the tunour suppressor protein p53 and this interaction results in a decrease in the half-life of p53 within cells (Wemess, B. A. et al (1990) Science. 248, 76-79, Scheffner, M., et al., (1990) Cell. 63, 1129-1136, Lechner, M. S. et al (1992) EMBO J., 11, 3045-3052, Hubbert, N. L. et al (1992) J. Virol., 66. 6237-6241). Since p53 can block cell cycle progression and/or induce apoptosis (Gottlieb, T. M. and Oren, M. (1998) Seminars in Cancer Biol., 8. 359-368), decreased levels of E6 might be expected to lead to increased levels of p53 and increased levels of cell cycle arrest and/or cell death (shown schematically in FIG. 1). In keeping with this view, expression of BPV1 E2 in HeLa cells appears to stabilise p53 (Hwang, E. S. et al (1993) J. Virol. 67, 3720-3729, Desaintes, C., et al. (1997) EMBO J., 16. 504-514). However, E2-TR also represses transcription of E6 and E7 in these cells, but this truncated E2 protein can neither stabilise p53, nor induce apoptosis (Desaintes, C., et al. (1997) EMBO J. 16. 504-514). This suggests that the N-terminal transcription regulation domain is responsible for these effects and that the repression of E6 transcription by E2 is not the critical event for the induction of apoptosis. In addition, the expression of HPV 31 E2 in NHK cells appears to de-stabilise p53 (Frattini. M. G. et al (1997) EMBO J. 16. 318-331).

The E7 protein binds to the Rb tumour suppressor protein and the Rb-related proteins p107 and p130 (Dyson, N., et al (1989) Science, 243, 934-937, Hu, T., et al (1995) Int. J. Oncology. 6. 167-174). The binding of E7 to Rb brings about the release of E2F proteins from Rb-E2F complexes and is also thought to target Rb for ubiquitin-dependent proteolysis (Boyer. S. N. et al (1996) Cancer Res. 56. 4620-3624, Jones, D. L. and and Munger, K. (1997) J. Virol., 71, 2905-2919, Virology, 239, 97-107). When released from Rb, members of the E2F family of transcription factors activate the transcription of genes required for S-phase and the over-expression of E2F-1 can induce apoptosis in serum-starved cells (Wu X., and Levine, A. J. (1994) Proc. Natl. Acad. USA 91. 3602-3606, Qin. X. Q., et al. (1994) Proc. Natl. Acad. Sci. USA. 91. 10918-10922). The repression of E7 transcription by E2 might therefore be expected to reduce the levels of free E2F, leading to cell cycle arrest (see the accompanying FIG. 1).

Expression of BPV1 E2 protein in HeLa cells is accompanied by decreased levels of E2F-1 mRNA and protein, and by reduced expression of E2F-dependent genes (Hwang, E. S. et al (1996) Oncogene, 12. 795-803). However, expression of the HPV 16 E2 protein in SiHa cells is accompanied by increased E2F activity (Sanchez-Perez, A. M. et al (1997) J. Gen. Virol. 78. 3009-3018). Furthermore, over-expression of the HPV 31 E2 protein in NHK cells is accompanied by an increase in E2F-1 mRNA levels (Frattini. M. G. et al (1997) EMBO J. 16. 318-331). Another complication is that unlike BPV1 E2, which represses transcription, the HPV16 and HPV18 E2 proteins have both been shown to activate transcription of the HPV16 E6 and E7 oncogenes (Bouvard, V., et al (1994) EMBO J 13 5451-5459, Kovelman, R., et al (1996) Virol, 70. 7549-7560). Any increase in the levels of E7 might be expected to result in increased levels of free E2F and this could in turn lead to cell death (Sanchez-Perez, A. M. et al (1997) J. Gen. Virol. 78. 3009-3018).

Models that seek to explain the effects of E2 proteins on cell proliferation via transcrptional repression or activation of the E6 and E7 genes, are obviously limited to HPV-positive cells. However, the HPV31 E2 protein appears to induce apoptosis in HPV-negative NHK cells (Frattini, M. G. et al (1997) EMBO J., 16. 318-331). Moreover, mutations in the HPV16 LCR that block the binding of E2 to the promoter proximal E2 sites and prevent E2-mediated repression of E6 and E7 transciption, do not fully relieve the negative effects of E2 on transformation efficiency (Romanczuk, H., and Howley. P. M. (1992) Proc. Natl. Acad. Sci. USA. 89., 3159-3163). These reports suggest that E2 might influence cell proliferation independently of its effects on the transcription of E6 and E7. To address this issue we have expressed the HPV16 E2 protein in a variety of transiently transfected cell lines. We have shown that this E2 protein can unexpectedly induce apoptosis in both HPV-transformed and non-HPV-transformed cell lines. In addition we have shown that the HPV 16 E2-induced apoptosis is p53-dependent and that the DNA binding activity of this E2 protein is not required for the induction of cell death.

WO98/01148 (Harvard) discloses methods and compositions for interfering with the proliferation of cells infected with and/or transformed by PV. There is no disclosure of the use of E2 to kill PV-negative cells, the p53 status of the cells, the induction of apoptosis in the treated cells, or the generation of an immune response to PV.

WO94/04686 (Biogen) describes a method for the delivery of proteins, including HPV E2 polypeptides, to cells based on the HIV TAT protein. There is no disclosure of the use of E2 to kill HPV-negative cells, the p53 status of the cells, the induction of apopthsis in the treated cells, or the generation of an immune response to PV.

WO92/12728 (Biogen) discloses non-functional E2-derived polypeptides, specifically E2 trans-activation repressors, which dimerise with normal E2 and block its function in HPV-infected cells. There is no disclosure of the use of E2 to kill PV-negative cells, the p53 status of the cells, the induction of apoptosis in the treated cells, or the generation of an immune response to PV.

WO98/32861 (Pasteur) discloses methods and compositions for interfering with the proliferation of cells infected with and/or transformed by PV. There is no disclosure of the use of E2 to kill PV-negative cells or the generation of an immune response to PV, no disclosure of the use of E7 to kill cells, and no disclosure of the use of any E2 proteins defective in DNA binding.

WO98/05248 (Bristol-Myers Squibb Company) discloses the use of polypeptides corresponding to peptides expressed in mammalian cells in response to PV infection and where the peptides correspond to part of the E6 or E7 proteins. There is no disclosure of using E2 peptides or E2 DNA sequences to vaccinate against PV infection or cervical cancer.

According to one aspect of the invention there is provided a method of inducing apoptosis of PV negative p53 wild-type, or p53 mutant-, or p53-related gene positive cells comprising contacting those cells with a PV E2 and/or E7 protein or a functional portion or derivative thereof or supplying to the cells a DNA sequence encoding a PV E2 and/or E7 protein or a functional portion or derivative thereof.

According to another aspect of the invention, there is provided a method of killing PV positive cells comprising contacting those cells with a PV E2 and/or E7 protein or a functional portion or derivative thereof or supplying to the cells a DNA sequence encoding a PV E2 and/or E7 protein or a functional portion or derivative thereof.

According to a further aspect of the invention, there is provided a method of killing cells infected with a non-HPV oncogenic virus comprising contacting those cells with a PV E2 and/or E7 protein or a functional portion or derivative thereof or supplying to the cells a DNA sequence encoding a PV E2 and/or E7 protein or a functional portion or derivative thereof.

According to a further aspect of the invention, there is provided a method of killing oncogenic cells or oncogenic precursor comprising contacting those cells with a PV E2 and/or E7 protein or a functional portion or derivative thereof or supplying to the cells a DNA sequence encoding a PV E2 and/or E7 protein or a functional portion or derivative thereof.

According to a further aspect of the invention there is provided a method of treating cervical cancer comprising contacting cervical cells of a subject with E2 and/or E7 or a functional portion thereof or supplying to the cells a DNA sequence encoding a PV E2 and/or E7 protein or a functional portion thereof. Such a method is advantageous in that an immune response against HPV may be produced by the E2 derivative in addition to causing death of the cancer cells.

According to a further aspect of the invention there is provided a method of inducing apoptosis of PV negative cells comprising contacting those cells with a PV E2 and/or E7 protein or a functional portion or derivative thereof and wild-type p53 protein or a functional portion or derivative thereof.

According to a further aspect of the invention there is provided a method of inducing apoptosis of PV positive cells comprising contacting those cells with a PV E2 and/or E7 protein or a functional portion or derivative thereof and wild-type p53 protein or a functional portion or derivative thereof. Such a method is advantageous in that an immune response raised against HPV may be produced by the E2 derivative.

According to a further aspect of the invention there is provided a method of inducing apoptosis of PV oncogenic cells comprising contacting those cells with a PV E2 and/or E7 protein or a functional portion or derivative thereof and wild-type p53 protein or a functional portion or derivative thereof.

According to a further aspect of the invention there is provided a method of inducing apoptosis of PV cervical cancer cells comprising contacting those cells with a PV E2 protein or a functional portion or derivative thereof and wild-type p53 protein or a functional portion or derivative thereof. Such a method is advantageous in that an immune response against HPV may be produced by the E2 derivative.

According to a further aspect of the invention there is provided a method of inducing apoptosis of PV negative cells comprising contacting those cells with a PV E2 and/or E7 protein or a functional portion or derivative thereof and wild-type P53 protein.

According to a further aspect of the invention there is provided a method of inducing apoptosis of PV negative cells comprising contacting those cells with a PV E2 and/or E7 protein or a functional portion or derivative thereof and wild-type P53 protein and/or drugs that induce wild-type p53 or wild-type p53 function in cells containing mutant p53.

According to a further aspect of the invention there is provided a method of inducing apoptosis of PV negative cells comprising contacting those cells with a PV E2 and/or E7 protein or a functional portion or derivative thereof and wild-type P53.

According to a further aspect of the invention there is provided a method of inducing apoptosis of PV negative cells comprising contacting those cells with a PV E2 ard/or E7 protein or a functional portion or derivative thereof and wild-type P53 protein and optionally agents that activate p53 function such as DNA damaging drugs, or UV, X-ray or other forms of radiation.

E2 or E7 or derivatives may be supplied by viral e.g adenovirus, adeno-associated virus, and pox virus, or non-viral methods such as VP22, penetratin, and liposomes.

In this invention, the use of E2 protein and functional derivatives or portions thereof are preferred although the use of E7 protein and functional derivatives or portions thereof are contemplated.

The use of DNA binding defective E2 derivatives is especially preferred in methods in accordance with the invention because they would not allow or participate in viral replication but would still kill cells and induce immune response.

The E2 proteins from HPV16, HPV18, and BPV1 all affect the proliferation and/or survival of cervical carcinoma cell lines (Sanchez-Perez, A. M. et al (1997) J. Gen. Virol., 78. 3009-3018, Desaintes, C. et al (1997) EMBO J. 16. 504-514, Hwang, E. S., et al (1993) J. Virol. 67. 3720-3729). We previously proposed that the HPV16 E2 protein induces apoptosis in HPV16-transformed SiHa cells by activating transciption of the viral E7 gene (Sanchez-Perez, A. M. et al (1997) J. Gen. Virol. 78. 3009-3018). In contrast, others have proposed that the HPV18 E2 protein induces apoptosis in HPV18-transformed HeLa cells by repressing transcription of the viral E6 and E7 genes (Desaintes, C., et al (1997) EMBJO J. 16. 504-514). Here we have shown that the HPV16 E2 protein induces apoptosis in several non-HPV-transformed cell lines, supporting the demonstration that the HPV 31 E2 protein appears to induce apoptosis in HPV-negative NHK cells (Frattini, M. G. et al (1997) EMBO J. 16 318-331). These data show that neither of the above mechanisms can be entirely correct. Either the E2 proteins induce apoptosis independently of the HPV genome or these proteins induce apoptosis via two pathways: one requiring other HPV proteins and one independent of other HPV proteins.

The E7 protein has been extensively studied, primarily as an oncoprotein, but also as an inducer of apoptosis. For example, E7 has been shown to sensitise keratinocytes to undergo both spontaneous apoptosis and apoptosis in response to tumour necrosis factor α (Stoppler, H. et al (1998) Oncogene. 17. 1207-1214). There is evidence to suggest that E7-induced apoptosis occurs via both p53-dependent and p53-independent pathways (Howes, K. A. et al (1994) Genes and Dev. 8, 1300-1310, Pan. H., and Griep. A. E. (1994) Genes and Dev. 8 1285-1299, Stoppler. H. et al (1998) Oncogene 17, 1207-1214). Here we show that in HeLa cells, over-expression of the HPV16 E7 protein induces apoptosis. The E7-induced-apoptosis can be abrogated by over-expression of the HPV 16 E6 protein, or by expression of a trans-dominant negative mutant of p53. These findings strongly suggest that the HPV16 E7 protein induces p53-dependent apoptosis in these cells. We have shown that, like E7, the HPV16 E2 protein induces apoptosis in HeLa cells and that this apoptosis is p53-dependent. Both E7 and E2 induce apoptosis in cells that contain the HPV16 E6 gene. Given that E6 binds to p53 and that this interaction results in a decrease in the half-life of p53, this might seem remarkable. However, p53 activity has been demonstrated in several HPV-positive cell lines (Butz, K., et al (1995) Oncogene, 10, 927-936, Desaintes, C., et al (1997) EMBO J. 16. 504-514).

We show that the sequence-specific DNA binding activity of the HPV16 E2 protein is not required for the induction of apoptosis in HeLa cells. In contrast, the N-terminal domain, including the transcrption activation domain and the hinge region, is essential for the promotion of cell death. Growth arrest by HPV1 E2 requires a functional DNA binding domain and a functional transcription activation domain (Goodwin, E. C. et al (1998) J. Virol. 72. 3925-3934). These data suggest that the induction of apoptosis by the HPV16 E2 protein and the induction of growth arrest by the BPV1 E2 protein, are brought about by two separate pathways. Growth arrest brought about by the BPV1 E2 protein is thought to require transcriptional regulation of the integrated HPV oncogenes. Thus, BPV1E2-induced growth arrest could be the result of transciptional repression of the integrated E6 and E7 genes (Goodwin, E. C. et al (1998) J. Virol. 72. 3925-3934). Repression of E6 transciption by BPV E2 would be expected to result in increased levels of p53 and this could lead to p53-dependent apoptosis (Hwang, E. S. et al (1996) Oncogene 12. 795-803, Desaintes, C. et al (1997) EMBO J. 16. 504-514). In contrast, the induction of apoptosis by HPV16 E2 occurs independently of its DNA binding activity and independently of the presence of integrated HPV sequences.

We show that two functional ascription activation domains are required for the induction of apoptosis by the HPV 16 E2 protein. Similar heterodimer experiments with BPV1 E2 and BPV1 E2-TR showed that two functional activation domains are required for efficient transcription activation (Barsoum, J. et al (1992) J. Virol. 66, 3941-3945). Why two activation domains per E2 dimer should be essential for either transcription activation, or the induction of apoptosis, is not presently known.

In short we show that the HPV16 E2 protein brings about apoptosis in the absence of other HPV gene products and that this E2-induced apoptosis is p53-dependent. Integration of the HPV genome into the host chromosome and the consequent disruption of the E2 gene removes this pro-apoptotic signal. Since the integrated HPV sequences continue to produce the E6 and E7 proteins, these cells continue to proliferate and are likely to form cervical tumours.

In this specification, the following expressions are used with the following, non-limiting meanings, given by of explanation:

1) “PV positive” means that a cell has been infected or transformed by PV and “PV negative” has an opposite meaning.

2) “Protein” and “polypeptide” are used interchangably although “protein” may generally be considered to be a naturally-occuring polypeptide.

Functional derivatives of E2 or E7 are proteins or polypeptides having at some of a biological function of E2 or E7, for example, the N-terminal transcription/replication domain of the E2 protein (amino acids 1-200) or the C-terminal DNA binding domain of the E2 protein (amino acids 279-365). Functional portions of E2 or E7 are peptides having at least some of native E2 or E7 sequence respectively.

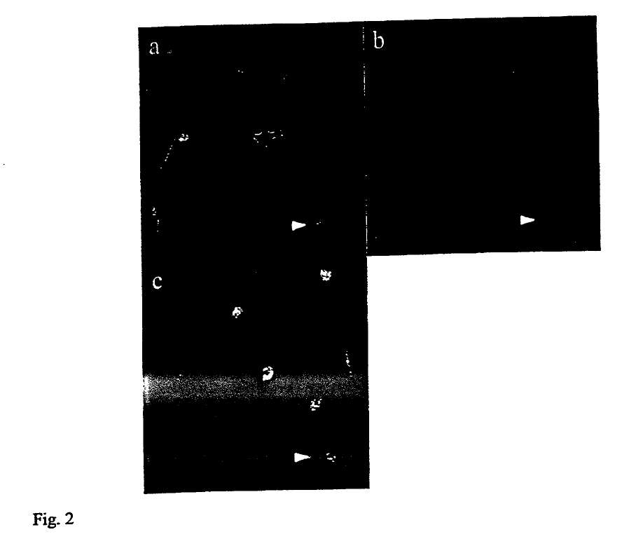

Methods of inducing cell death and products in accordance with the invention will now be described, by way of example only, with reference to the further accompanying drawings, FIGS. 2 to 14 in which:

FIG. 2 shows the effect of HPV 16 E2 on HeLa cells.

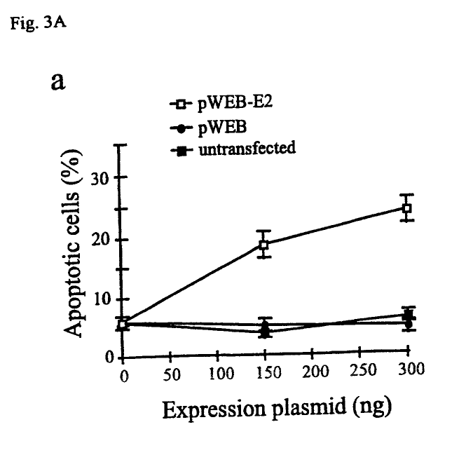

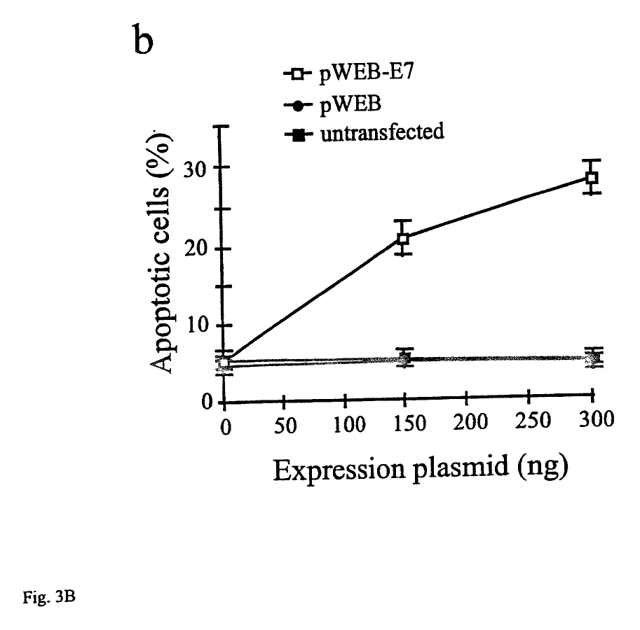

FIG. 3 shows the results of experiments using E2 and E7 to induce apoptosis in HeLa cells.

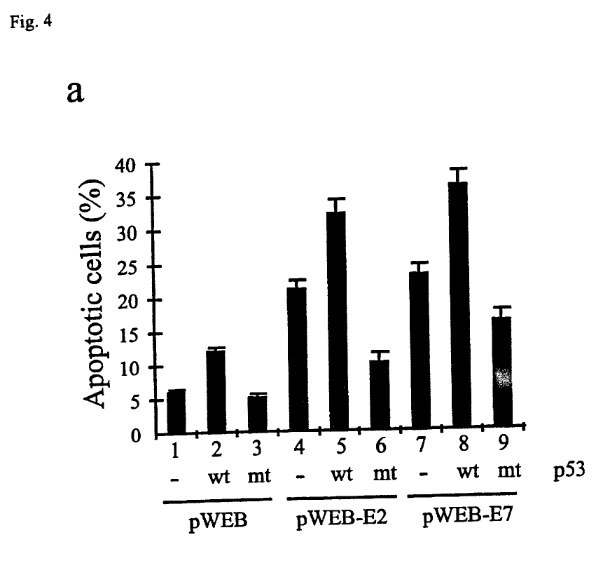

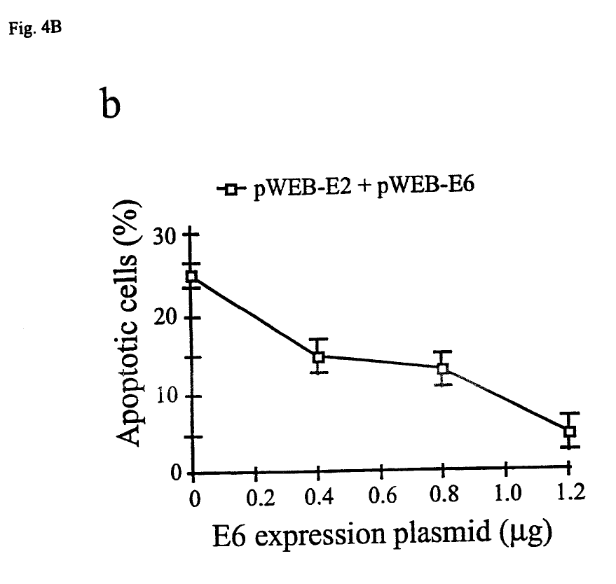

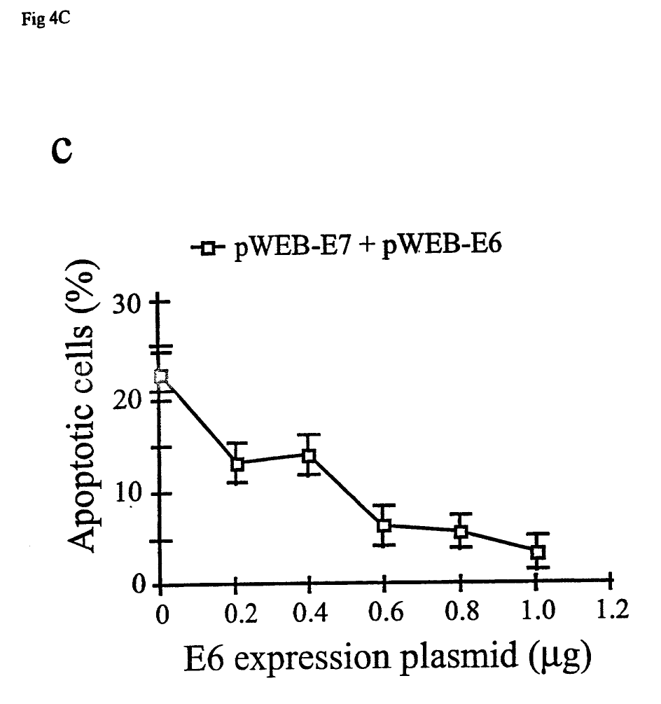

FIG. 4 shows the results of experiments which demonstrate that E2- and E7-induced apoptosis is p53-dependent;

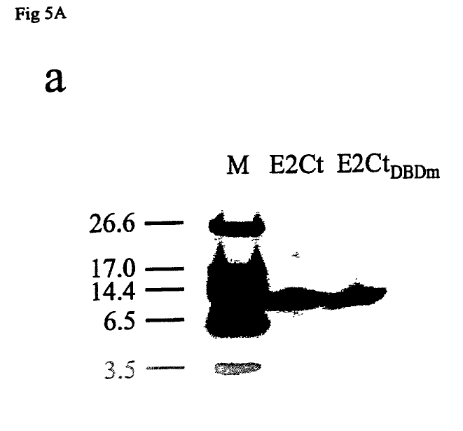

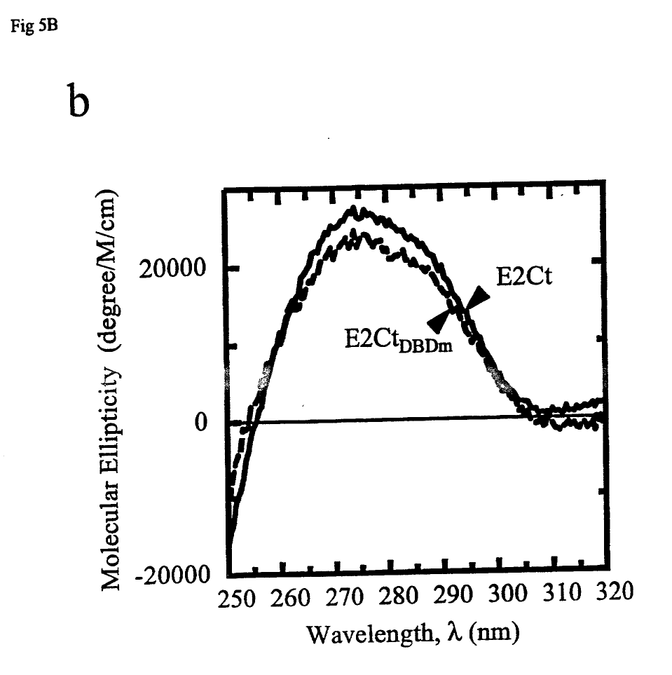

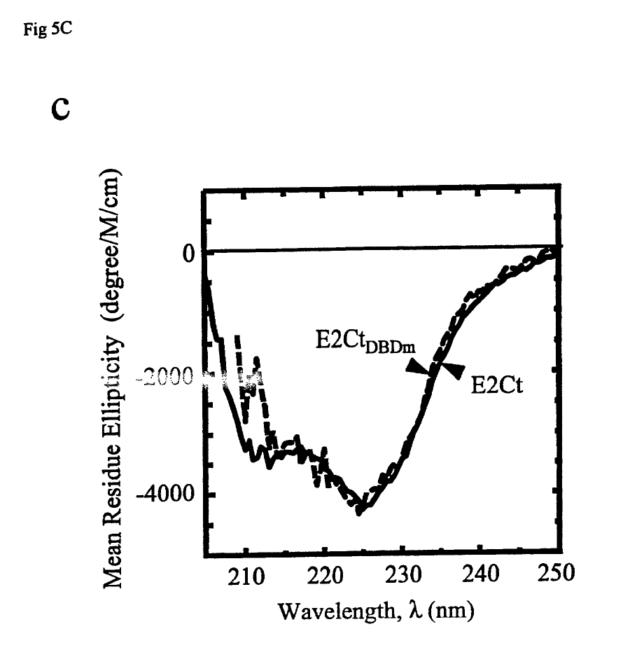

FIG. 5 shows that results of experiments which show that the truncated E2 protein E2Ct mutated at N296, K299, and R304 folds and dimerises but fails to bind DNA;

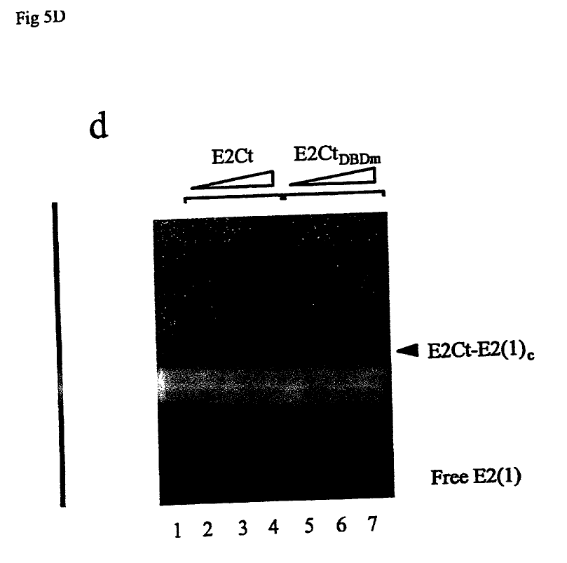

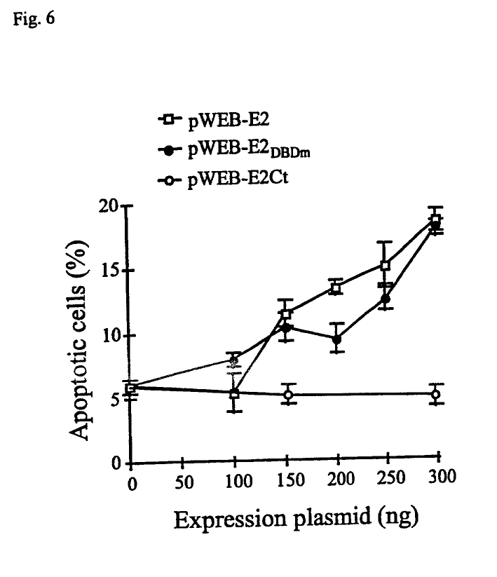

FIG. 6 shows the results of experiments which show that DNA binding is not required for the induction of apoptosis, but that the N-terminal transcription regulation domain is required;

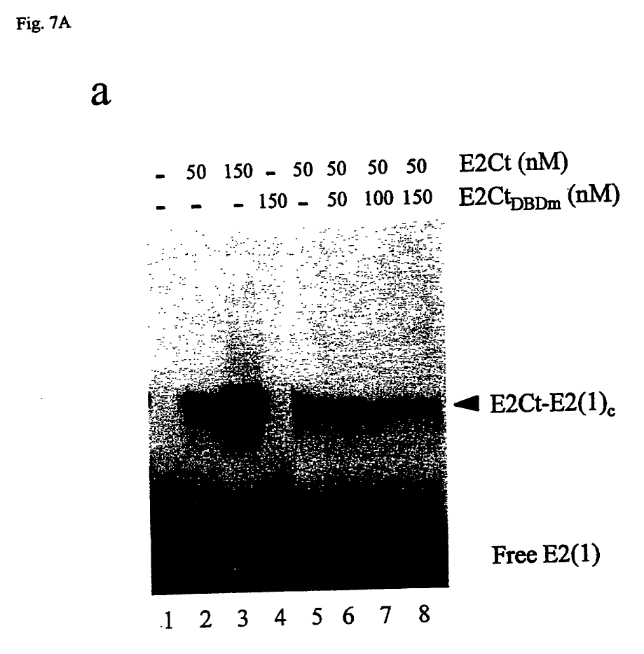

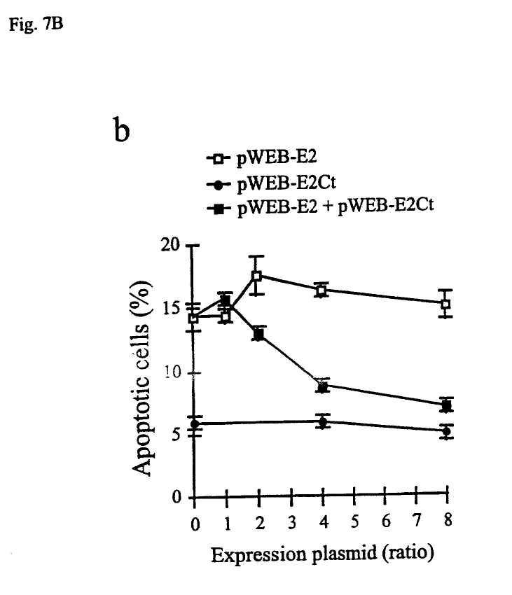

FIG. 7 shows the results of experiments which demonstrate that E2-E2Ct heterodimers do not induce apoptosis; and

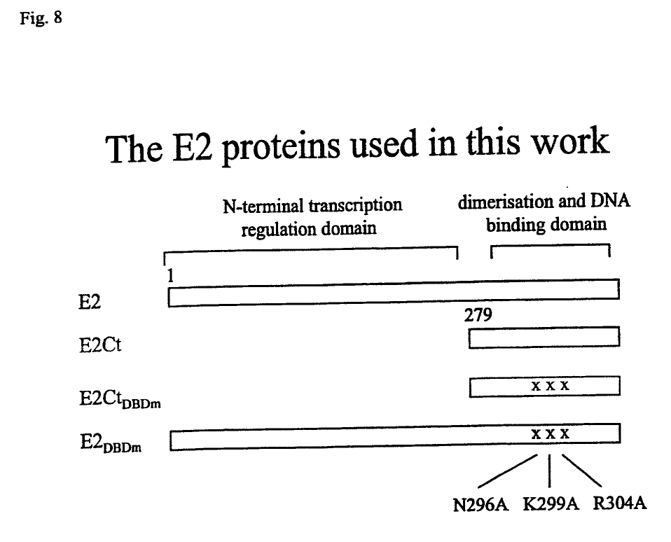

FIG. 8 shows schematically the proteins used in the following experiments;

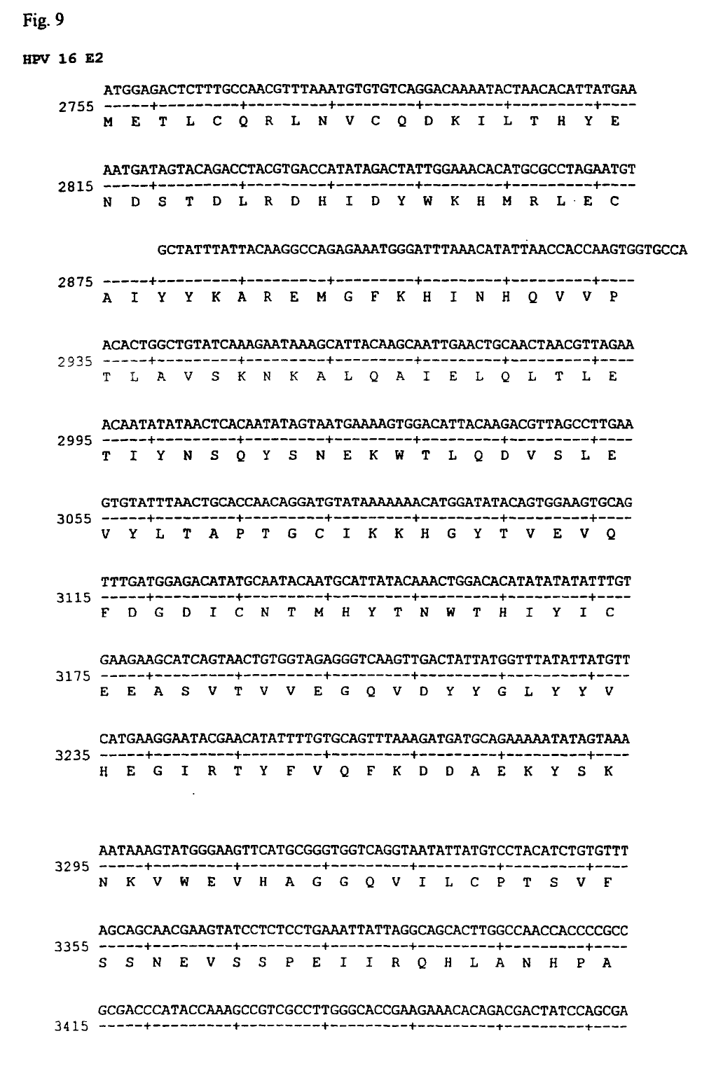

FIG. 9 shows the DNA and amino acid sequences of HPV16E2;

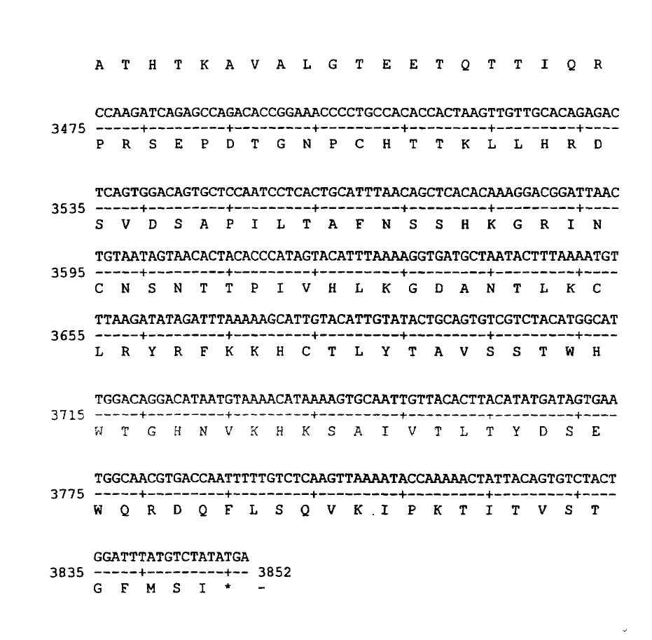

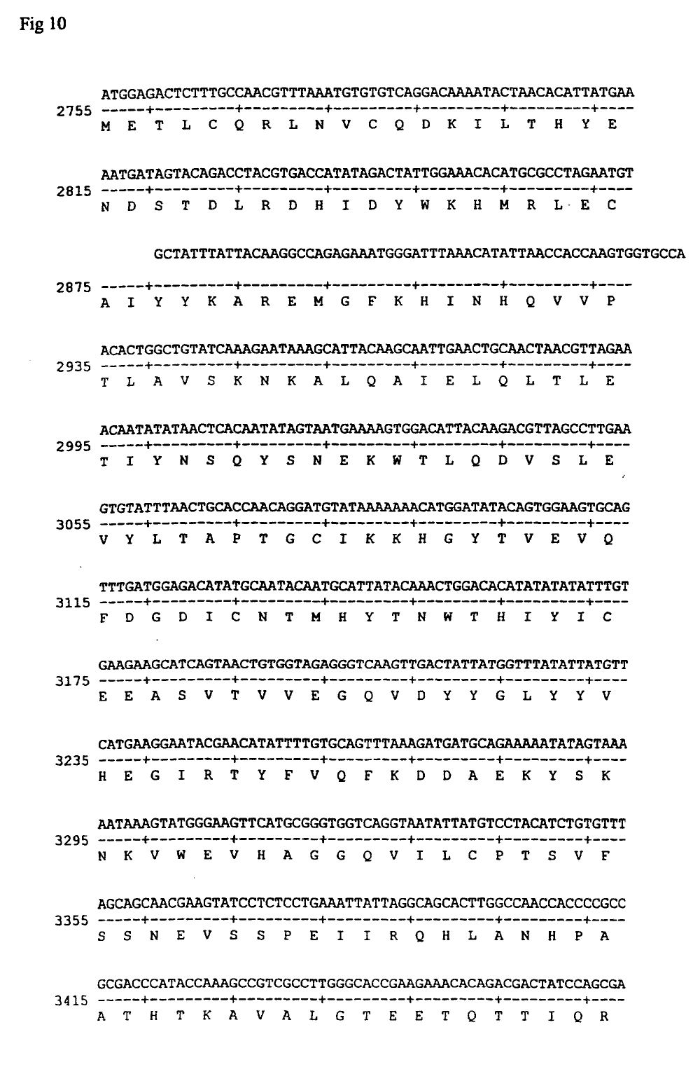

FIG. 10 shows the DNA and amino acid sequences of HPVE2DBM;

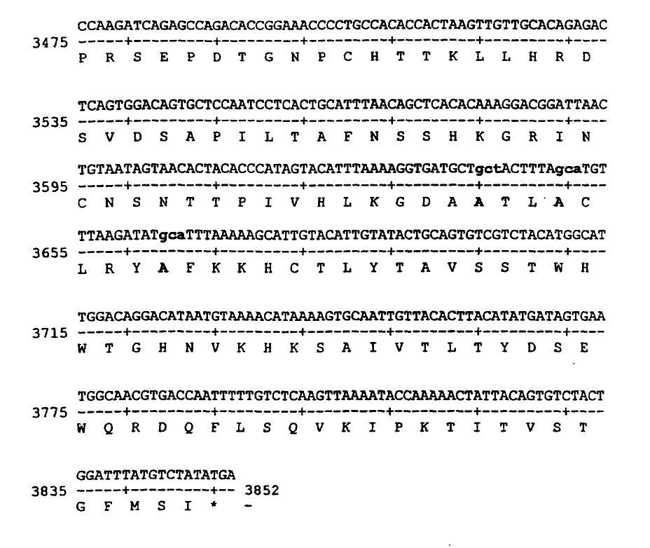

FIG. 11 shows the DNA and amino acid sequences of E2Ct;

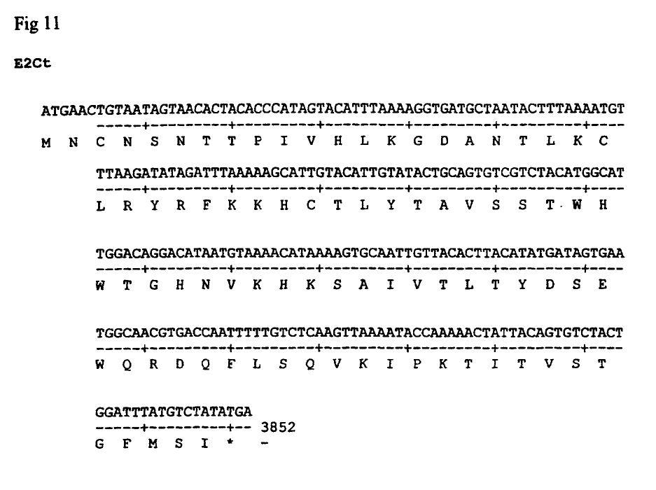

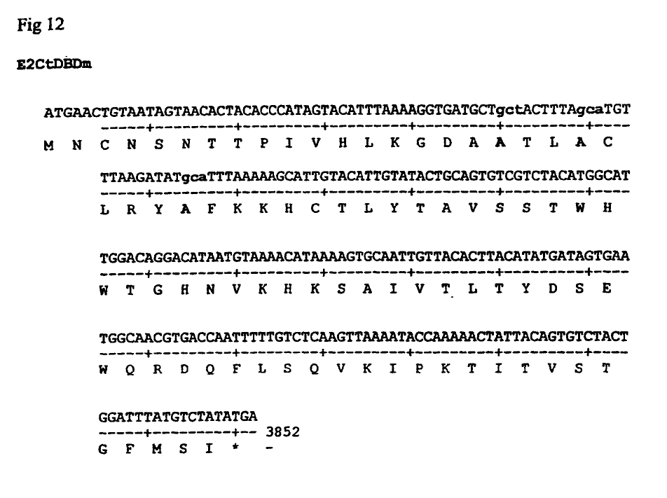

FIG. 12 shows the DNA and amino acid sequence of E2CtDBm.

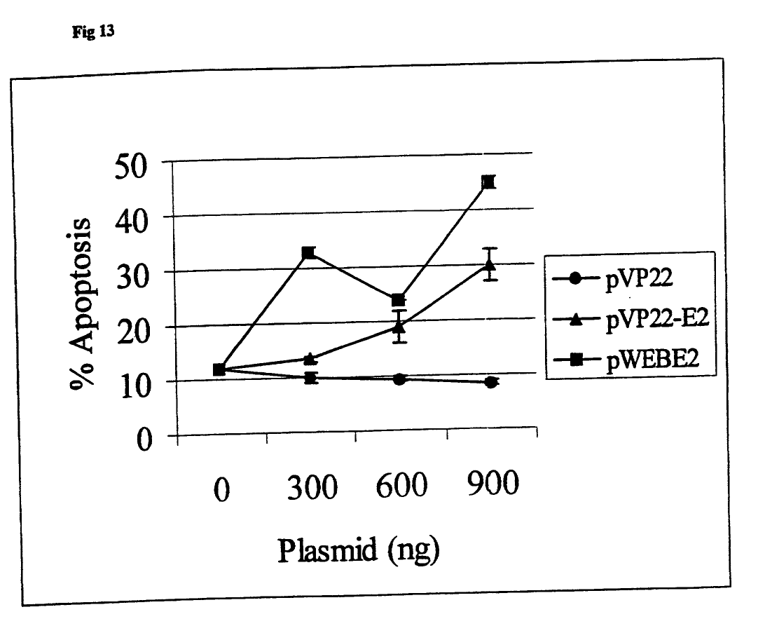

FIG. 13 shows that a VP22-E2 fusion protein induces apoptosis in HeLa cells.

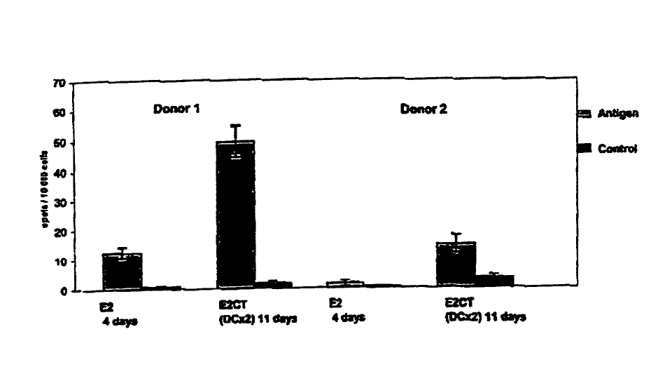

FIG. 14 shows HPV16 E2 specific T cell responses.

1. Experimental Procedures—General

a. Plasmids.

The plasmids pCB6+p53 and pCB6+p53173L express wild-type and mutant p53, respectively, and were supplied by Dr Moshe Oren and Dr Andy Phillips. Plasmid pCMX-GFP3 expresses green fluorescent protein and was supplied by Dr Jeremy Tavaré. The pWEB plasmid was made by removing an XhoI-EcoRI fragment carrying the CMV promoter from pUHD 15-1 and using this fragment to replace the tetracycline-inducible promoter in pUHD10-3. The HPV16 E2 (FIG. 9), E6, and E7 expression plasmids were produced by cloning the appropriate HPV sequences obtained from HPV 16 genomic DNA into a unique Eco RI site in pWEB, immediately downstream of the CMV promoter.

The E2 gene was amplified by PCR (94° C. for 1 minute, 53° C. for 3 minutes, and 72° C. for 1 minutes, for 30 cycles) from HPV 16 DNA template using the oligonucleotide primers E25′ 5′ CTACGAATTCATGGAGACTCTTTGCCAACG 3′ and E23′ 5′GATAGAATTCTCATATAGACATAAATCCAG 3′. These primers place EcoRI restriction sites (highlighted in bold throughout) at the 5′ and 3′ ends of the E2 coding sequence. The PCR product was cloned into the Eco RI site of pWEB and sequenced using a panel of E2-specific sequencing primers to check for the occurrence of any point mutations.

The E6 gene was amplified by PCR (95° for 1 minute, 52° for 1 minute, and 68° C. for 2 minutes, for 30 cycles) from HPV 16 template using the prinmers E65′ 5′TGAGAATTCATGCACCAAAAGAGAACTGCAATGTTTCAG 3′ and E63′ 5′ATCGAATTCTTACAGCTGGGTTTCTCTACG 3′ which have Eco RI sites. The PCR product was cloned into the EcoRI site of pWEB and sequenced using a panel of E6-specific sequencing primers.

The E7 gene was amplified by PCR (94° C. for 1 minute, 54° C. for 2 minutes, and 72° C. for 1 minute, for 30 cycles) from a HPV 16 template using the primers E75′ 5′TCGGAATTCATGCATGGAGATACACCTAC3′ and E73′ 5′ AGCGAATTCTTATGGTTTCTGAGAACAGATGG 3′ which have Eco RI sites. The PCR product was cloned into pWEB and sequenced using the E7 PCR primers.

Mutated E2 constructs were generated using PCR. The plasmid pWEB-E2DBDm expresses a mutated E2. protein in which three amino acids within the E2 DNA binding domain (N296, K299, and R304) have been replaced by alanines. The mutations were introduced by PCR (94° C. for 1 minute, 55° C. for 1 minute, and 68° C. for 1 minute, for 30 cycles) using the primers pWEB5′ 5′ ACCTCCATAGAAGACACCGGG 3′ and E2m 5′CGACACTGCAGTATACAATGTACAATGCTTTTTAAATGCATATCTTAAACATGCTAAAGTAGCAGCATCACC 3′ with pWEB-E2 as template. The bases in italics mismatch the E2 gene and introduce the mutations. The PCR product contains a PstI site (highlighted in bold) at its 3′ end. This site, and an SstI site located within the CMV promoter, were used to replace the wild-type E2 sequence in pWEB-E2 with the mutated E2DBDm sequence. The entire PCR product was sequenced using a panel of E2-specific sequencing primers to check for the occurrence of any unwanted mutations.

The plasmid pWEB-E2Ct expresses a truncated E2 protein that lacks the N-terminal amino acids of E2 from 1 to 279 but dimerises and binds DNA normally (Lewis, H., and Gaston, K. (1999) J. Mol. biol. 294: 885-896). To create this mutant, HPV 16 sequences between base pairs 3592 and 3852 were amplified by PCR (94° C. for 1 minute, 55° C. for 1 minute, and 68° C. for 1 minute, for 30 cycles) using the primers E2Ct5′ 5′GAAACAGAATTCATGAACTGTAATAGTAACACTACACCC 3′ and E23′ with pWEB-E2 as template. These primers place EcoRI restriction sites (bold) at both ends of the product and introduce an ATG translation start codon (italics). The PCR product was cloned into the EcoRI site in pWEB and sequenced using E2-specific primers. The plasmid pWEB-E2CtDBDm expresses a DNA binding defective version of E2Ct. This plasmid was produced exactly as described for pWEB-E2Ct except that pWEB-E2DBDm (which comprises full length E2 with the N296A K299A and R304A mutations) was used as template in the PCR reaction.

The 86 amino acid E2Ct (FIG. 11) and E2CtDBDm (FIG. 12) proteins were expressed in Escherichia coli XL1-blue cells using expression vector pKK223-3 (Pharmacia Biotech). The sequences encoding E2Ct and 2CtDBDm were excised as EcoRI fragments from pWB-E2Ct and pWEB-E2CtDBDm, respectively, and cloned into a unique EcoRI site downstream of the Ptac promoter in pKK223-3. The inserts were sequenced using E2 and pKK223-3-specific primers.

The plasmid pVP22-E2 was created by cloning the entire HPV16 E2 open reading frame into the multiple cloning site of plasmid pVP22/Myc-His (Invitrogen) in frame with the VP22 open reading fame. The insert was sequenced using pVP22/Myc-His-specific primers.

b. Protein Purification and Circular Dichroism Spectroscopy.

E. coli XL1-blue cells Stratagene containing either pKK-E2Ct or pKK-E2CtDBDm were grown to an OD600 nm of 0.5. Protein expression was then induced with 1 mM IPTG and the cells incubated at 37° C. overnight. The cells were harvested by centrifugation, resuspended in 50 mM Tris-Acetate-EDTA buffer (pH 7.5) containing 1 mM MgCl2 and 1% 2-mercaptoethanol, and then lysed by sonication at 4° C. The cell lysate was cleared by centrifugation (15,000 g for 30 minutes at 4° C.) then incubated with 0-1% DNase I for 30 minutes at 20° C. The cell extract was dialysed for three hours against 50 mM phosphate buffer (pH 5.7) containing 1% 2-mercaptoethanol and then re-centrifuged. The supernatant was loaded onto an S-Sepharose cation exchange medium column equilibrated in 50 mM phosphate buffer (pH 5.7) containing 10 mM DTT. After washing with 50 column volumes of phosphate buffer, the E2 protein was eluted using a linear gradient of 0.2-1M NaCl in the same buffer over 500 ml (at 1 ml/minute). Protein peaks (detected by A²⁸⁰ nm) were collected and analysed by SDS-PAGE and gel retardation assays (data not shown). Pooled E2 fractions were dialysed against 10 volumes of 50 mM phosphate buffer (pH 5.7) containing 10 mM DTT for three hours and then applied to a MonoS HR 16/10 cation exchange FPLC column equilibrated in the same buffer. E2 was eluted with a 0.1-1M NaCl gradient and dialysed against 25mM Na phosphate buffer (pH 7.9) containing 1 mM DTT for 3 hours before being snap frozen and stored at −70° C. Isolelectric points (pI) and molecular weight values were determined from the amino acid sequences of wild-type (pI 9.7; Mr 10016.6) and mutant E2 (pI 9.4; M, 9831.4) using Expasy Tools. Molecular weights were confirmed on a VG Quattro triple quadrupole mass spectrophotometer with electrospray ionization. Structural integrity was confirmed using far-UV and near-UV circular dichroism spectroscopy on a Jobin Yvon CD6 spectropolarimeter using a 0.05 cm path length with a 0.5 nm resolution at 1 nm/minute.

c. Gel Retardation Assays.

A double stranded oligonucleotide (100 ng) corresponding to the sequence of the HPV 16 E2 site 1 from nucleotides 46 to 65 (5′TTGAACCGAAACCGGTTAGT 3′) was end labelled with [γ-³²P] ATP using T4 polynucleotide kinase (Stratagene). Unincorporated label was removed using a Sephadex G-50 column (Stratagene). Labelled oligonucleotides (10000 cpm) were incubated with purified proteins in binding buffer (20 mM HEPES (pH 7.9), 25 mM KCl, 1 mM DTT, 0.1% NP-40, 10% glycerol, 0.5 μg/μl bovine serum albumin, 80 ng/μl poly[d(I-C)]). After 20 minutes at 20° C., free and bound labelled DNA were resolved on 6% non-denating polyacrylamide gels run in 0.5×TBE and visualised by autoradiography. Heterodimers between wild-type E2Ct and E2CtDBDm were formed by mixing and denaturing the proteins in 3M urea (1 hour at 20° C.) and then refolding by dilution to 0.1M urea in binding buffer. The DNA binding activity of the heterodimers was assayed exactly as descibed above.

d. Cell Culture and Transfections.

SiHa, C33a, Saos-2, MCF-7, and COS-7 cells were maintained in Dulbecco's Modified Eagles Medium (DMEM: Sigma) supplemented with 10% Foetal Bovine Serum (FBS: Sigma) and penicillin (100000 U/liter) and streptomycin (100 mg/liter). NIH 3T3 cells were maintained in DMEM supplemented with 10% Calf Serum (CS:Sigmna) and penicillin/streptomycin. HeLa cells were maintained in Minimal Essential Medium (MEM: Sigma) supplemented with 10% FBS, 2 mM L-glutamine and penicillin/streptomycin. 866, 873, 877, 915 and 808F cells were maintained in DMEM supplemented with 5% FBS, penicillin/streptomycin, 2 mM L-glutamine, 5 μg/ml insulin, 0.01 μg/ml EGF, 0.01 μg/ml cholera toxin and 0.4 μg/ml hydrocortisone. All cells were maintained at 37° C. in 5% CO₂.

Prior to transient tranfection, cells were seeded at 3×10⁵ cells per well onto coverslips in 6 well plates and incubated overnght to obtain a sub-confluent culture. The liposome based reagents Tfx-50, for SiHa and NIH3T3 cells, and Tfx-20, for all other cell lines, (Promega) were used at a 3:1 liposome:DNA ratio in 1 ml serum free media per transfection, according to the manufacturer's instructions. The DNAs used in each transfection are described in the text. After 18 hours (E7 experiments) or 30 hours (E2 experiments), the coverslips were washed in PBS and the cells fixed with 4% paraformaldehyde/PBS at 22° C. for 30 minutes. Following further washes with PBS, the cells were stained with bisbenzimide (Hoechst No.33258: Sigma) for 30 minutes before being washed in PBS and mounted onto microscope slides in 10 μl MOWIOL (Calbiochem).

e. Fluorescence Microscopy and Imaging.

Fluorescence microscopy was carried out using a Leica DM IRBE inverted epi-fluorescent microscope fitted with FITC and DAPI filter sets and a 20× air objective (Leica). Imaging was carried out using a Leica DM IRBE inverted confocal microscope using a 63× oil objective (Leica) and TCS-NT4 software (Leica).

2. The HPV 16 E2 and E7 Proteins Induce Apoptosis in HeLa Cells.

The plasmids pWEB-E2, pWEB-E6, and pWEB-E7 express the HPV16 E2, E6 and E7 proteins, respectively. Each of these plasmids was transiently transfected in increasing amounts into HeLa cells growing on coverslips using liposomes FIG. 2 shows a representative group of HeLa cells visualised using a 40× oil immersion lens fitted to an epi-fluorescent microscope: (a) Bright field microscopy. (B)GFP fluorescence. (c) DAPI fluorescence. In each experiment the GFP-expressing plasmid pCMX-GFP3 was co-transfected into the cells; pCMX-GFP3 expresses the green fluorescent protein (GFP) and allows transfected cells to be identified by their fluorescence upon excitation through an FITC filter set. Since GFP is expressed uniformly throughout the transfected cell it also allows the assessment of cellular morphology (FIG. 2b). The cells were stained with bisbenzimide (Hoechst stain) which enters the nuclei of all of the cells, regardless of their transfection status, and allows comparison of chromatin condensation between untransfected cells and transfected cells within the population (FIG. 2c). Individual cells were scored as untransfected or transfected using GFP and assessed for chromatin condensation and membrane blebbing using Hoechst stain and GFP, respectively. A typical transfected cell that is undergoing apoptosis is indicated in FIG. 2. Membrane blebbing is seen in (a) and (b). Chromatin condensation is seen in (c). The percentage of untransfected cells and transfected cells undergoing apoptosis was determined by counting.

The percentage of apoptotic HeLa cells seen after transient transfection with the E2, and E7 expressing plasmids is shown in FIG. 2. In each experiment around 5% of the untransfected cells, and around 5% of the cells transfected with the empty pWEB vector, are apoptotic. Both the E2 and the E7 expression plasmids a significantly increased the levels of apoptosis within the transfected population (FIG. 3a and FIG. 3b, respectively). Apoptotic cells in the transfected and untransfected populations were identified as shown in FIG. 2. The transfection was performed in duplicate and repeated three times. These data show that the HPV16 E2 and E7 proteins both induce apoptosis in HeLa cells. We next set out to determine whether these protein can induce apoptosis in other cell lines.

3. E2 and E7 Induce Apoptosis in Both HPV-transformed and Non-HPV-transformed Cell Lines.

We assayed the ability of the HPV 16 E2 and E7 proteins to induce apoptosis in 6 HPV-transformed cell lines and 4 non-HPV transformed cell lines. The results of this comparison are shown in Table 1.

Table 1E2 and E7 induce apoptosis in a variety of cell lines.

| | | Apoptotic Cells (%)^{#} | | |
|---|---|---|---|---|
| Cell Line | HPV status | Background* | HPV 16 E2 | HPV 16 E7 |
| HeLa | HPV 18 | 5 ± 2 | 26 ± 3 | 27 ± 3 |
| SiHa | HPV 16 | 5 ± 2 | 44 ± 3 | 46 ± 3 |
| 866 | HPV 16 | 5 ± 2 | 22 ± 2 | 22 ± 2 |
| 873 | HPV 18 | 7 ± 2 | 35 ± 3 | 24 ± 2 |
| 877 | HPV 18, 45 | 6 ± 2 | 28 ± 3 | 28 ± 3 |
| 915 | HPV 16 | 7 ± 2 | 33 ± 3 | 23 ± 2 |
| C33a | — | 8 ± 2 | 9 ± 2 | 8 ± 3 |
| COS-7 | — | 6 ± 2 | 6 ± 2 | 5 ± 2 |
| 808F | — | 6 ± 2 | 26 ± 3 | 20 ± 3 |
| NIH3T3 | — | 6 ± 2 | 25 ± 3 | 20 ± 2 |
| Saos-2 | — | 11 ± 2 | 12 ± 2 | 12 ± 2 |
| MCF-7 | — | 5 ± 2 | 60 ± 3 | 52 ± 3 |
| | | (20 ± 2) | | |

^{#}The percentage of apoptotic cells was determined as described in the text.
*The background level of apoptosis refers to the untransfected population and the population transfected with the empty pWEB plasmid. These values are the same except in the case of MCF-7 cells in which the percentage apoptosis after pWEB transfection is given in brackets.

MCF-7 transfected with pWEB showed higher background levels of apoptosis.

E2 and E7 induced of apoptosis in HeLa cells and SiHa cells, an HPV18- and an HPV16-transformed cervical carcinoma cell line, respectively. Both E2 and E7 also induced of apoptosis in human 866, 873, 877, and 915 keratinocytes: 866 cells and 915 cells contain HPV16, 873 cells contain HPV18, and 877 cells contain both HPV18 and HPV45 (Bartholomew, J. et al (1997) Cancer Res. 57, 937-942 and P. Stern, unpublished observations). Interestingly, both E2 and E7 failed to induce apoptosis in either C33a cells, COS-7 cells or Saos-2 cells, a non-HPV-transformed cervical carcinoma cell line, an SV40-transformed monkey fibroblast cell line and a human osteosarcoma cell line, respectively. However, E2 and E7 did induce high levels of apoptosis in three other HPV-negative cell lines: 808F cells, NIH3T3 cells and MCF-7 cells, a human fibroblast cell line, a mouse fibroblast cell line and a human breast carcinoma cell line, respectively. Thus, E2 is capable of inducing apoptosis in HPV-negative cell lines. Another striking feature of these results is that all the cell lines induced to undergo apoptosis by E2, are also induced to undergo apoptosis by E7. Similarly, the cell lines that are not sensitive to E2 expression, are not sensitive to E7 expression. Thus E2 and E7 induce apoptotic cell death via the same pathway, or via pathways that converge at some point.

All of the cell lines that were seen to undergo apoptosis in response to E2 or E7 expression are thought to contain wild-type p53. For example, NIH 3T3 cells contain wild-type p53 and can undergo p53-dependent apoptosis (Chirillo, P. et al (1997) Proc. Natl. Acad. Sci. USA. 94. 8162-8167). In contrast, C33a cells contain mutated p53 (Crook. T., et al (1991) Oncogene. 6 873-875) and Saos-2 cells are p53-null and both these cell lines fail to undergo apoptosis in response to either E2 or E7. To determine whether p53 plays a role in E2 and/or E7-induced cell death, we next looked at the effects of a trans-dominant negative p53 mutant and expression of the HPV16 E6 protein, on the levels of apoptosis in cells expressing these proteins.

4. E2 and E7 Induce Apoptosis Via a p53-dependent Pathway.

HeLa cells were transiently transfected with the GFP-expressing plasmid pCMX-GFP3 and either pWEB, pWEB-E2, or pWEB-E7 and either pCB6+p53, which expresses wild-type p53 (wt), or pCB6+p53173L, which expresses mutant p53 (mt). Apoptotic cells were identified as in FIG. 2.

Co-expression of wild-type p53 increased the level of apoptosis induced by both E2 and E7 by almost 50% (compare columns 4 and 5 and 7 and 8 in FIG. 4a). In contrast, co-expression of the trans-dominant negative p53173L mutant decreased the level of apoptosis induced by both E2 and E7 to near the basal level (columns 6 and 9 respectively in FIG. 4a). These data suggest that under these conditions the apoptosis induced by the HPV 16 E2 and E7 proteins occurs through a p53-dependent pathway. To confirm this conclusion, HeLa cells were transiently co-transfected with pWEB-E2 or pWEB-E7 and either pWEB-E6, or the empty pWEB vector. The HPV16 E6 protein binds p53 in conjunction with the E3 ubiquitin ligase enzyme E6-AP and this results in the degradation of p53 via a ubiquitin-dependent protease (Scheffner, M. et al (1990) Cell. 63. 1129-1136, Huibregtse, J. M. et al (1991) EMBO J. 10. 4129-4135). The addition of increasing amounts of the pWEB-E6 plasmid resulted in a gradual decrease in the level of E2-induced apoptosis (FIG. 4b). Similarly, the level of apoptosis induced by the E7 protein was also significantly reduced by the co-expression of E6 (FIG. 4c). In the presence of large amounts of pWEB-E6, the levels of both E2- and E7-induced apoptosis were reduced to around the basal level. Taken together these results firmly establish that functional p53 is required for apoptosis induced by both the HPV16 E2 protein, and the HPV16 E7 protein.

5. The DNA Binding Activity of E2 is not Required for the Induction of Apoptosis.

Although there are several plausible mechanisms whereby E7 over-expression could result in apoptosis, the route from E2 over-expression to apoptosis is unclear. We originally proposed that in SiHa cells, E2 might increase transcription of the integrated E7 oncogene, and that this might result in E7-induced cell death. However, here we have shown that E2 can induce apoptosis in several non-HPV-transformed cell lines (Table 1). These data imply that E2 does not kill cells simply by activating transcription of E7. To confirm this hypothesis we placed thee point mutations within the E2 DNA binding domain (DBD) at positions known to be important for protein-DNA interactions. The crystal structures of the HPV16 E2 DBD and the BPV1 E2 DBD-DNA complex suggest that amino acids N296, K299, and R304 within the HPV16 E2 DBD, are critical for the recognition of specific E2 binding sites (Hegde, R. S. and Androphy. E. J. (1998) J. Mol. Biol. 284. 1479-1489, Hegde, R. S. et al (1992) Nature 359. 505-512). Using site-directed mutagenesis we replaced all three of these amino acids with alanines. The mutations were introduced in the context of both the full length E2 protein, and the E2 DNA binding domain alone.

To establish that these mutations abolish DNA binding activity without disrupting the overall folding of the E2 DBD, we expressed both the wild-type DBD and the mutated DBD in bacteria. The plasmid pKK-E2Ct expresses a truncated E2 protein (amino acids 280 to 365) that can dimerise and bind DNA normally (Lewis H. and Gaston. K (1999) J. Mol. Biol. 294: 885-896). The plasmid pKK-E2CtDBDm expresses the equivalent E2 fragment containing the N296A, K299A, and R304A mutations. The E2Ct and E2CtDBDm proteins were purified from bacteria carrying the respective plasmids (FIG. 5a).

Specifically, in the FIG. 5(a) experiment samples of purified E2Ct and E2CtDBDm were analysed by SDS-PAGE. The sizes of the markers used are indicated in the Figure. In the FIGS. 5(b) and (c) experiment circular dichroism was used to show that the presence of the N296, K299, and R304 mutations did not affect the folding or dimerisation of E2CtDBDm. (d) increasing amounts (10, 50, and 250 nM, respectively) of E2Ct (lanes 2-4) or E2CtDBDm (lanes 5-7) were added to labelled oligonucleotides carrying E2 binding site1 from the HPV16 genome: E2(1). Free and bound DNA was separated on a 6% polyacrylamide gel and visualised by autoradiography. The E2Ct-E2(1) complex is indicated by an arrowhead. Circular dichroism (CD) was then used to test whether the presence of the mutations altered the folding or dimerisation of the E2 DBD. The CD spectra for the E2Ct and E2CtDBDm proteins (FIGS. 5b and 5c) are very similar. This implies that the mutations have little or no effect on these properties. FIG. 5d shows the results of a gel retardation assay in which increasing amounts of the E2Ct protein (lanes 2-4) or the E2CtDBDm protein (lanes 5-7) were added to labelled oligonucleotides carrying an E2 binding site. As can be seen from the Figure, E2Ct binds tightly to the labelled DNA whereas E2CtDBDm exhibits no detectable binding to this site.

To determine whether the DNA binding activity of E2 is required for the induction of cell death, we transiently transfected HeLa cells with plasmid pCMX-GFP3 expressing either the wild-type full length E2 protein (pWEB-E2), or full length E2 carrying the N296A, K299A, and R304A mutations (pWEB-E2DBDm). Apoptotic cells were identified as shown in FIG. 2. The transfection was performed in duplicate and repeated three times. Somewhat surprisingly, the pWEB-E2 and pWEB-E2DBDm plasmids induced almost identical levels of cell death (FIG. 6). In contrast, the plasmid pWEB-E2Ct, which expresses the E2 DBD alone and therefore lacks the N-terminal transcription activation domain, failed to induce cell death (FIG. 6). Thus, although the sequence-specific DNA binding activity of E2 is not required for the induction of apoptosis in HeLa cells, the N-terminal transcription activation domain is indispensable. To confirm and extend these conclusions we next looked at the ability of E2 heterodimers to induce cell death.

6) Two Functional N-terminal Domains are Required for E2-induced Cell Death.

The BPV1 E2 and E2-TR proteins have previously been shown to form heterodimers (Barsoum, J. et al (1992) J. Virol. 66. 3941-3945). Although these heterodimers are reported to bind DNA in vitro, they fail to activate transcription in intact cells (Barsoum, J. et al (1992) J. Virol. 66. 3941-3945). In view of this, we wanted to determine whether the HPV 16 E2 and E2Ct proteins would form heterodimers and whether these heterodimers would be capable of inducing cell death. To ascertain whether or not heterodimers could be formed in vitro, we mixed a fixed amount of wild-type E2Ct, with increasing amounts of the DNA binding defective E2CtDBDmn protein. The amounts of E2Ct and E2CtDBDm indicated in FIG. 7a were mixed and then denatured in 3M urea to facilitate the exchange of subunits. After refolding by dilution into 0.1M urea, the proteins were added to labelled oligonucleotides carrying the HPV16 E2 binding site1: E2(1). Free and bound DNA was separated on a 6% polyacrylamide gel and visualised by autoradiography. The E2Ct-E2(1) complex is indicated by an arrow. Re-folded E2Ct binds to a labelled oligonucleotide carying an E2 site whereas re-folded E2CtDBDm shows no DNA binding activity (FIG. 7a, lanes 3 and 4, respectively). Adding increasing amounts of E2CtDBDm to a fixed amount of E2Ct resulted in a gradual decline in DNA binding activity (FIG. 7a, lanes 5-8). These data show that at least in this in vitro assay, these E2 proteins can form heterodimers.

To investigate the formation of heterodimers in intact cells, HeLa cells were transiently transfected with pWEB-E2 and increasing amounts of the empty pWEB plasmid (open squares), pWEB-E2Ct and increasing amounts of pWEB (filled circles), or pWEB-E2 and increasing amounts of pWEB-E2Ct (filled squares). Apoptotic cells were identified as in FIG. 2 and the transfection was performed in duplicate and repeated three times. The pWEB-E2 plasmid induced high levels of cell death in the transfected population whereas the pWEB-E2Ct plasmid had no effect (FIG. 7b). However, as increasing amounts of the pWEB-E2Ct plasmid were added to a transfection mixture containing pWEB-E2, the percentage of apoptotic cells in the transfected population declined and eventually reached background levels (FIG. 7b). Increasing amounts of the pWEB-E2CtDBDm plasmid also decreased the level of pWEB-E2-induced cell death whereas increasing amounts of pWEB had no effect (not shown). These data suggest that heterodimers containing E2 and E2Ct form in intact cells and that these heterodimers are incapable of inducing apoptosis. Thus it appears that the E2 dimer requires two functional N-terminal domains in order to induced cell death.

7. A VP22-E2 Fusion Protein can Induce Apoptosis.

The Herpes Simple Virus type 1 (HSV-1) protein VP22 is a 38kDa protein found in the tegument region of the virion, between the capsid and the envelope. When expressed in a transiently transfected cell, VP22 is transported into the cytoplasm and is then exported from the cell of synthesis via a non-classical secretion mechanism. The protein then enters the surrounding cells with very high efficiency, and is localised to the nucleus by a mechanism that is dependent on the actin cytoskeleton. Once inside the nucleus, VP22 binds chromatin and is segregated into the daughter cells. VP22 transport between cells is so efficient that the protein can enter every cell in a transfected monolayer (Elliott, G and O'Hare, P (1997) Cell 88: 223-233).

In order to clone the E2 ORF into the VP22 vector (Invitrogen) in the correct reading frame, it was first removed from the plasmid pWEBE2 and inserted into the multiple cloning site of pBluescript II KS (Stratagene) as an EcoR1 fragment. This construct was then digested with EcoRV and BamHI and the resulting E2 fragment was inserted into the pVP22 multiple cloning site. DNA sequence analysis was performed using primers specific for both pVP22 and E2.

The plasmids pVP22-E2, pVP22, and pWEB-E2 were co-transfected into HeLa cells with pCMX-GFP3 and the percentage of apoptotic cells in the transfected and untransfected populations was determined exactly as described previously. After transient transfection with pVP22-E2 the percentage of apoptotic cells increases to around 30% (FIG. 13). In contrast, the pVP22 plasmid has no effect on the level of apoptosis. These data show that the VP22-E2 fusion protein is capable of inducing apoptosis in HeLa cells.

8. E2 and the Immune Response

E2 and the Immune Response to HPV

Previous data are consistent with a possible role for E2 immunity in the control of HPV infection in cervical cancer and pre-malignant HPV-related disease (Rocha-Zavaleta et al., (1997) Brit. J. Cancer 75, 1144-1150). Induced immunity to E2 could be protective and would in principle target the earliest stages of viral expression in the basal/parabasal cells of the cervical epithelium. Stimulating local long lived mucosal immunity would have the advantage over capsid protein based vaccines in eliminating cells with DNA in episomal form. Cells with episomal HPV DNA might form the pool (latent state) from which integration events (a major risk factor for progression) may occur subsequently. PV positivity in women can be about 39% in sexually active women aged 15-25. This decreases markedly with age and supports the view that there is an acquired immunity to infection.

The prospect of local delivery of an immunogenic E2 protein or a modified E2 protein which can activate an apoptosis pathway would represent a complementary attack on any cervical lesion with both therapeutic and preventative components. This should be relevant to high risk and low risk virus infection including genital and other warts. An interesting possibility is that the immunogenicity of E2 protein may be enhanced by being bound to its target DNA. This complex might deliver unique epitopes which are recognised by the immune response in natural clearance.

We have shown that T helper responses to HPV 16 E2 appear to correlate with the clearance of viral infections emphasising the potential of this protein as a prophylactic and therapeutic immune target (Bontkes, H. J et al, (1999). Gen Virol 80: 2453-2459). We have now developed assays for measuring HPV 16 E2-specific T cell responses using γ interferon ELISPOT. FIG. 14 shows evidence of memory T cells versus HPV 16 E2 in donor 1 but not in donor 2. However, prolonged exposure to HPV 16 E2 C-terminus fragment with autologous dendritic cells prepared from peripheral blood monocytes is able to generate a primary activation of specific T-cells. We have prepared dentritic cells (DCs) from adherent peripheral blood mononuclear cells in serum free medium with GM-CSF and IL-4. The majority of cells are CD1a+, HLA-DR+, CD80+ and CD14−. Peripheral blood lymphocytes, depleted of CD4 cells were incubated with autologous DCs and 10 μg/ml of E2Ct (prepared as described in section 1b) or with no antigen for 4 or 11 days (responder cells were washed and fresh DCs and E2 or no antigen added at day 9). ELISPOT was performed following replating and overnight incubation. Spots were counted at three different cell concentrations in triplicate, data normalised and mean and SE calculated. These data support the possibility that E2 T cells with anti-HPV lesion activity can be generated and/or boosted in humans.

1. A method of inducing apoptosis of PV negative p53 wild-type, p53 mutant- or p53 related gene positive cells comprising contacting those cells with a PV E2 protein or a functional portion or derivative thereof or supplying to the cells a DNA sequence encoding a PV E2 protein or a functional portion or derivative thereof whereby apoptosis is induced in the cells.

2. A method of killing cells infected with an oncogenic virus comprising contacting those cells with a PV E2 protein or a functional portion or derivative thereof or supplying to the cells a DNA sequence encoding a PV E2 protein or a functional portion or derivative thereof whereby the cells are killed.

3. A method of killing PV negative oncogenic cells or oncogenic precursor cells comprising contacting those cells with a PV E2 protein or a functional portion or derivative thereof or supplying to the cells a DNA sequence encoding a PV E2 protein or a functional portion or derivative thereof whereby the cells are killed.

4. A method of inducing apoptosis of PV negative or PV positive cells comprising contacting those cells with a PV E2 protein or a functional portion or derivative thereof and wild-type p53 protein or a functional portion or derivative thereof whereby apoptosis is induced in the cells.

5. A method of inducing apoptosis of oncogenic cells comprising contacting those cells with a PV E2 protein or a functional portion or derivative thereof and a wild-type p53 protein or a functional portion or derivative thereof whereby apoptosis is induced in the cells.

6. A method of inducing apoptosis of PV positive cervical cancer cells comprising contacting those cells with a PV E2 protein or a functional portion or derivative thereof and wild-type p53 protein or a functional portion or derivative thereof whereby apoptosis is induced in the cells.

7. A method of inducing apoptosis according to any one of claims 1 to 6 comprising contacting cells with a) PV E2 protein, and b) wildtype p53 protein and/or drugs or pharmaceutical compositions that induce wildtype p53 or wildtype p53 function in cells containing mutant p53.

8. A method of inducing apoptosis in HPV negative cells according to any one of claims 1 to 5 comprising contacting cells with a PV E2 protein and at least one agent that activates p53 function.

9. A method according to any preceding claim in which a substantial proportion of cells in a treated cell population are killed.

10. A method according to any one of claims 4 to 9 in which the cells are infected with HPV.

11. A method according to claim 10 in which the cells are transformed by HPV.

12. A method according to claim 10 or 11 in which the HPV is integrated in the cell genome.

13. A method according to claim 10, 11, or 12 in which the HPV is type 16 and/or 18.

14. A method according to any preceding claim in which the cells are mammalian cells.

15. A method according to claim 14 in which the cells are for example cervical, breast, lung, brain, colon, oesphagus, melanoma, osteosarcoma, skin, liver, head, carcinoma, and leukeamia cells.

16. A method according to claim 15 in which the cells are in a mammalian subject

17. A method according to claim 15 or 16 in which the cells are human cells.

18. A method according to any one of claims 1, 2 or 9 to 17 in which the cells are oncogenic cells or precursors thereof.

19. A method according to any preceding claim in which the cells are virally infected.

20. A method according to claim 19 in which the cells are infected with an oncogenic virus or retrovirus.

21. A method according to claim 20 in which the oncogenic virus is an HPV, Hepatitis B, Herpesvirus B, Epstein-Barr virus or Human T cell lymphotropic virus type 1 or type 2.

22. A method according to claim 19 in which the cells are infected with HIV or HIV-related virus.

23. A method according to any preceding claim in which an immune response, preferably a mucosal response is induced in the cells or subject.

24. A method according to claim 23 in which the immune response is a mucosal response.

25. A method according to any preceding claim in which the cells over-express p53 or a related gene or a mutant thereof.

26. A method according to claim 25 in which the p53 related gene is p63 or p73.

27. A method according to any preceding claim in which the E2 derivative or functional portion binds DNA less well than wildtype E2.

28. A method of treating cervical cancer comprising contacting cervical cells of a subject with E2 or a functional portion thereof or supplying to the cells a DNA sequence encoding a PV E2 protein or a functional portion thereof.

29. A method according to claim 28 in which the subject is human.

30. A method according to claim 28 or 29 including inducing an immunological response in a subject against PV.

31. A method according to claim 23, 25, 28, 29 or 30 in which E2 is fused to another protein or functional portion thereof which elicits an immune response.

32. A method according to claim 32 in which the other protein or portion thereof is tetanus toxoid fragment C.

33. A method according to any preceding claim in which a DNA sequence encoding a portion or derivative of E2 is used in which the portion or derivative is defective in DNA binding compared to wild type E2.

34. A method according to any preceding claim in which the E2 protein or functional portion or derivative thereof is complexed with DNA.

35. A method according to any one of claims 1 to 34 in which the PVE2 derivative is a DNA binding-defective E2 derivative comprising an E2 amino acid sequence lacking C terminal portion(s) of a native E2 sequence.

36. A method according to claim 35 in which the PVE2 derivative is a DNA binding-defective E2 derivative according to claim 35 comprising an E2 sequence in which the last 86 amino acids of the native E2 protein are missing.

37. A method according to claim 35 or 36 in which the PVE2 derivative is a DNA binding-defective E2 derivative comprises an E2 sequence in which amino acids 296, 299 and 304 of the native E2 protein are missing.

38. A homodimer comprising a DNA binding-defective E2 derivative comprising an E2 amino acid sequence lacking C terminal portion(s) of a native E2 sequence; or a DNA binding-defective E2 derivative according to claim 35 comprising an E2 sequence in which the last 86 amino acids of the native E2 protein are missing; or a DNA binding-defective E2 derivative comprises an E2 sequence in which the amino acids 296, 299 and 304 of the native E2 protein are missing.

39. A heterodimer comprising a DNA binding-defective E2 derivative comprising an E2 amino acid sequence lacking C terminal portion(s) of a native E2 sequence; or a DNA binding-defective E2 derivative according to claim 35 comprising an E2 sequence in which the last 86 amino acids of the native E2 protein are missing; or a DNA binding-defective E2 derivative comprises an E2 sequence in which the amino acids 296, 299 and 304 of the native E2 protein are missing.

40. A vector comprising a modified E2 DNA sequence or at least a portion of an E2 sequence in which the E2 DNA sequence encodes an E2 derivative which can kill p53 positive cells, PV positive cells, cells infected with an oncogenic virus or oncogenic cells.

41. A vector according to claim 40 which encodes a polypeptide which will generate an immune response in a mammalian cell.

42. A vector according to claim 41 in which a mucosal response is generated.

43. A vector according to claim 40, 41, or 42 in which the E2 DNA sequence encodes an E2 derivative which can inhibit viral replication.

44. A vector according to claim 43 in which the derivative will inhibit replication of HPV.

45. A vector according to claim 40, 41, 42, 43 or 44 in which the E2 DNA sequence encodes an E2 derivative which is defective in binding DNA compared to wild type E2.

46. A nucleotide sequence encoding an E2 derivative for use in a method according to claim 36.

47. A nucleotide sequence according to claim 46 in which the sequence is a DNA or RNA sequence.


SEQUENCE LISTING (8 sequences)

SEQ ID NO: 1 - protein, 365 aa, Human papillomavirus
METLCQRLNVCQDKILTHYENDSTDLRDHIDYWKHMRLECAIYYKAREMGFKHINHQVVPTLAVSKNKALQAIELQLTLETIYNSQYSNEKWTLQDVSLEVYLTAPTGCIKKHGYTVEVQFDGDICNTMHYTNWTHIYICEEASVTVVEGQVDYYGLYYVHEGIRTYFVQFKDDAEKYSKNKVWEVHAGGQVILCPTSVFSSNEVSSPEIIRQHLANHPAATHTKAVALGTEETQTTIQRPRSEPDTGNPCHTTKLLHRDSVDSAPILTAFNSSHKGRINCNSNTTPIVHLKGDANTLKCLRYRFKKHCTLYTAVSSTWHWTGHNVKHKSAIVTLTYDSEWQRDQFLSQVKIPKTITVSTGFMSI

SEQ ID NO: 2 - DNA, 1098 bp, Human papillomavirus
atggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaagtggtgccaacactggctgtatcaaagaataaagcattacaagcaattgaactgcaactaacgttagaaacaatatataactcacaatatagtaatgaaaagtggacattacaagacgttagccttgaagtgtatttaactgcaccaacaggatgtataaaaaaacatggatatacagtggaagtgcagtttgatggagacatatgcaatacaatgcattatacaaactggacacatatatatatttgtgaagaagcatcagtaactgtggtagagggtcaagttgactattatggtttatattatgttcatgaaggaatacgaacatattttgtgcagtttaaagatgatgcagaaaaatatagtaaaaataaagtatgggaagttcatgcgggtggtcaggtaatattatgtcctacatctgtgtttagcagcaacgaagtatcctctcctgaaattattaggcagcacttggccaaccaccccgccgcgacccataccaaagccgtcgccttgggcaccgaagaaacacagacgactatccagcgaccaagatcagagccagacaccggaaacccctgccacaccactaagttgttgcacagagactcagtggacagtgctccaatcctcactgcatttaacagctcacacaaaggacggattaactgtaatagtaacactacacccatagtacatttaaaaggtgatgctaatactttaaaatgtttaagatatagatttaaaaagcattgtacattgtatactgcagtgtcgtctacatggcattggacaggacataatgtaaaacataaaagtgcaattgttacacttacatatgatagtgaatggcaacgtgaccaatttttgtctcaagttaaaataccaaaaactattacagtgtctactggatttatgtctatatga

SEQ ID NO: 3 - protein, 365 aa, Human papillomavirus
METLCQRLNVCQDKILTHYENDSTDLRDHIDYWKHMRLECAIYYKAREMGFKHINHQVVPTLAVSKNKALQAIELQLTLETIYNSQYSNEKWTLQDVSLEVYLTAPTGCIKKHGYTVEVQFDGDICNTMHYTNWTHIYICEEASVTVVEGQVDYYGLYYVHEGIRTYFVQFKDDAEKYSKNKVWEVHAGGQVILCPTSVFSSNEVSSPEIIRQHLANHPAATHTKAVALGTEETQTTIQRPRSEPDTGNPCHTTKLLHRDSVDSAPILTAFNSSHKGRINCNSNTTPIVHLKGDAATLACLRYAFKKHCTLYTAVSSTWHWTGHNVKHKSAIVTLTYDSEWQRDQFLSQVKIPKTITVSTGFMSI

SEQ ID NO: 4 - DNA, 1098 bp, Human papillomavirus
atggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaagtggtgccaacactggctgtatcaaagaataaagcattacaagcaattgaactgcaactaacgttagaaacaatatataactcacaatatagtaatgaaaagtggacattacaagacgttagccttgaagtgtatttaactgcaccaacaggatgtataaaaaaacatggatatacagtggaagtgcagtttgatggagacatatgcaatacaatgcattatacaaactggacacatatatatatttgtgaagaagcatcagtaactgtggtagagggtcaagttgactattatggtttatattatgttcatgaaggaatacgaacatattttgtgcagtttaaagatgatgcagaaaaatatagtaaaaataaagtatgggaagttcatgcgggtggtcaggtaatattatgtcctacatctgtgtttagcagcaacgaagtatcctctcctgaaattattaggcagcacttggccaaccaccccgccgcgacccataccaaagccgtcgccttgggcaccgaagaaacacagacgactatccagcgaccaagatcagagccagacaccggaaacccctgccacaccactaagttgttgcacagagactcagtggacagtgctccaatcctcactgcatttaacagctcacacaaaggacggattaactgtaatagtaacactacacccatagtacatttaaaaggtgatgctgctactttagcatgtttaagatatgcatttaaaaagcattgtacattgtatactgcagtgtcgtctacatggcattggacaggacataatgtaaaacataaaagtgcaattgttacacttacatatgatagtgaatggcaacgtgaccaatttttgtctcaagttaaaataccaaaaactattacagtgtctactggatttatgtctatatga

SEQ ID NO: 5 - protein, 87 aa, Human papillomavirus
MNCNSNTTPIVHLKGDANTLKCLRYRFKKHCTLYTAVSSTWHWTGHNVKHKSAIVTLTYDSEWQRDQFLSQVKIPKTITVSTGFMSI

SEQ ID NO: 6 - DNA, 264 bp, Human papillomavirus
atgaactgtaatagtaacactacacccatagtacatttaaaaggtgatgctaatactttaaaatgtttaagatatagatttaaaaagcattgtacattgtatactgcagtgtcgtctacatggcattggacaggacataatgtaaaacataaaagtgcaattgttacacttacatatgatagtgaatggcaacgtgaccaatttttgtctcaagttaaaataccaaaaactattacagtgtctactggatttatgtctatatga

SEQ ID NO: 7 - protein, 87 aa, Human papillomavirus
MNCNSNTTPIVHLKGDAATLACLRYAFKKHCTLYTAVSSTWHWTGHNVKHKSAIVTLTYDSEWQRDQFLSQVKIPKTITVSTGFMSI

SEQ ID NO: 8 - DNA, 264 bp, Human papillomavirus
atgaactgtaatagtaacactacacccatagtacatttaaaaggtgatgctgctactttagcatgtttaagatatgcatttaaaaagcattgtacattgtatactgcagtgtcgtctacatggcattggacaggacataatgtaaaacataaaagtgcaattgttacacttacatatgatagtgaatggcaacgtgaccaatttttgtctcaagttaaaataccaaaaactattacagtgtctactggatttatgtctatatga